(19) United States
(12) Patent Application Publication
(10) Pub. No.: US 2009/0240202 A1
(43) Pub. Date: Sep. 24, 2009

(54) Expandable introducer sheath

(76) Inventors: William Joseph Drasler, Minnetonka, MN (US); Joseph Michael Thielen, Buffalo, MN (US)

Correspondence Address: William J. Drasler, 4100 Dynasty Drive, Minnetonka, MN, 55345, US

(21) Appl. No.: 12/381,700

(22) Filed: Mar. 16, 2009

Related U.S. Application Data
(60) Provisional application No. 61/070,397, filed on Mar. 21, 2008.
(60) Provisional application No. 61/070,398, filed on Mar. 21, 2008.

(51) Int. Cl. A61M 25/01

(52) U.S. Cl. 604/164.03

(57) ABSTRACT

An expandable introducer sheath can actively be induced to reduce in diameter or enlarge in diameter due to a braided internal structure that has axial strands connected to its distal end. The introducer sheath is delivered to the blood vessel or other tubular member of the body in a smaller diameter configuration with the axial strands in compression. The catheter is flexible and can be delivered to eccentrically shaped and tortuous vessels. Once the catheter is in position, the axial strands are placed into tension to cause the introducer sheath to assume a larger diameter configuration that will help straighten out tortuous vessels. The access site into the blood vessel is expanded via a radial expansion that is less traumatic. Passage of a larger interventional catheter can proceed through the larger diameter sheath configuration. If the interventional catheter has a smaller shaft, the introducer sheath can be reduced in diameter to come into close approximation with the shaft to reduce the opening size at the access site. The sheath can be removed over time with incremental reductions in its diameter to allow the access site time to relax to a smaller size for improved vascular sealing.

### CROSS REFERENCE TO RELATED APPLICATIONS

This invention makes reference and thereby includes aspects of provisional patent applications entitled Expandable Introducer Sheath with application No. 61/070,397 and entitled Rapid Exchange Guide Catheter with application No. 61/070,398, both filed 21 Mar. 2008 by Joseph M. Thielen.

### BACKGROUND OF THE INVENTION

1. Field of Invention

This invention relates to an interventional catheter that is placed within a blood vessel or other vessel of the body to provide access and support for another interventional catheter having passage within it and delivered to the site of a lesion.

2. Description of Prior Art

Access to coronary arteries, carotid arteries, the aorta, and peripheral vessels or other tubular members of the body for percutaneous therapeutic and diagnostic catheters is often made via guide catheters that are placed through introducer sheaths which are positioned into vessels that are most easily accessed from outside of the body. Such access sites include the common femoral artery and the radial artery. Other tubular members include the ureter, urethra, intestinal track, veins, and other tubular tissues of the body.

Typically a Seldinger approach is used to gain access to a blood vessel using a small needle through which is passed a guide wire and the needle is then removed. A dilator with an introducer sheath loaded upon it is then passed over the guidewire and into the artery. The dilator and guidewire are then removed leaving the introducer sheath in place and providing passage from outside the body to within the vessel lumen.

This method of vessel access works well in most cases, however when a vessel is similar in size or only slightly larger in comparison to the diameter of the introducer sheath, blockage of blood flow within the vessel can occur leading to thrombosis and potential compromise to the patient's limb. Also, vessels of the body can often be tortuous thus restricting the passage of therapeutic devices through them. Some therapeutic catheters are very large in diameter particularly at their distal portions. This is often the case for example with transfemorally placed percutaneous aortic valve catheter systems that have a larger profile distal portion and a smaller profile shaft. It is not favorable to use a large introducer sheath with a fixed large diameter to provide access to such catheters and expose the arterial access site to excessive trauma due to shear and extension for a long period of time. Tortuous anatomy can also preclude the use of large percutaneous interventional catheters due to inability to advance such catheters through the vasculature. This can often occur when attempting to access the heart with larger profile catheters from a transfemoral approach. Also, iliac arteries and other peripheral arteries can often be eccentric in shape thus not allowing passage of standard fixed diameter introducer sheaths. A device is needed to enable large profile catheters to be delivered easily and safely via a percutaneous procedure through tortuous vessels without creating excessive trauma to the arterial access site.

### SUMMARY

It is the purpose of the present invention to provide an improved introducer sheath that will allow larger therapeutic catheter devices to enter into a blood vessel or tubular member that is of a similar diameter and into vessels with tortuous anatomy. The introducer sheath will cause the tortuous vessel to become partially straightened and allow passage of an interventional catheter. The introducer sheath is delivered to the vessel in a smaller diameter configuration than a standard fixed diameter introducer sheath and expands out radially to provide an improved radial expansion of the access site. It also will cause eccentrically shaped vessels to become more rounded in cross-section. Therapeutic catheters that have a large distal aspect but with smaller diameter proximal shaft can be delivered with less trauma to the blood vessel and access site. The expandable introducer of the present invention enlarges in diameter to allow passage of this larger aspect through it, and can retract down to a smaller diameter that matches the smaller dimension of the proximal shaft of the therapeutic catheter. Vascular perfusion past the introducer is improved in comparison to current introducer sheaths. In addition, the vascular closure procedure that is performed following the therapeutic or diagnostic procedure is further improved due to exposure of the vasculature to a smaller diameter sheath for most of the procedure. The present expandable introducer sheath can also be reduced in diameter over a period of time following removal of the interventional catheter and completion of the interventional procedure. This would allow time for the access site puncture into the vessel to reduce in diameter and be more amenable to a percutaneous closure procedure.

One embodiment of the present invention is a passive expandable introducer that is a tubular structure having a braided fiber structure contained within that holds it in a smaller relaxed diameter but allows it to expand to a larger diameter as a larger aspect of a passing therapeutic or diagnostic interventional catheter is provided passage through the expandable introducer sheath. A polymeric coating is applied continuously along the braided tubular structure. A slippery coating is applied to the inner surface of the sheath to reduce friction with the interventional catheter to reduce binding of the sheath onto the outer surface of the interventional catheter. This introducer sheath changes diameter via a passive mechanism that occurs by the outward force of the larger diameter aspect of the passing catheter pushing the introducer outwards to a larger diameter.

This passively braided introducer sheath embodiment can also have a large diameter relaxed state and can be held into a smaller diameter configuration via application of axial tension during delivery. This tension can be applied from a dilator that holds onto the distal end of the introducer sheath during delivery. After it has been delivered into the vasculature, the introducer sheath can be released and allowed to expand to a larger diameter. The smaller diameter of the present sheath during delivery allows it to pass through tortuous vasculature that could not be passed with a standard introducer sheath.

In another embodiment the expandable introducer sheath can be made to perform a diameter change in an active manner. The introducer is constructed with a tubular structure formed of braided fibers and having axial strands that extend significantly in an axial direction along the introducer wall and attach to the distal end of the braided fibers. In one embodiment these axial strands can be placed under compression during the delivery of the introducer sheath to ensure that sheath remains in an elongated configuration with a smaller diameter during insertion or delivery into the body. This smaller diameter configuration is smaller than the diameter of a standard fixed diameter introducer sheath. This provides it with greater flexibility and ability to enter smaller tortuous or eccentrically shaped vessels. After the introducer sheath has been positioned within the blood vessel, tension can be placed on the axial strands to initiate a shape change in the introducer sheath from its equilibrium state having a longer length and a smaller diameter to a larger diameter state of shorter length. The braided fibers are brought into close approximation with each other thereby providing an increase in rigidity that assists in straightening out a tortuous blood vessel and providing improved passage for the interventional catheter. Such an expandable introducer sheath has direct application for placing a percutaneous aortic valve catheter system or other large interventional catheter from the femoral access site into the aorta, for example. The present invention can assist in reducing the tortuosity found in the iliac or femoral arteries and allow delivery of a larger catheter through tortuous and diseased iliac and other arteries.

An interventional catheter having a larger profile distal aspect can pass freely through the introducer after it has been expanded to a larger diameter. Once the distal aspect has passed through the expanded sheath, the axial strands can be released from tension if appropriate to allow the introducer to reduce in diameter to match the smaller proximal shaft diameter of the passing catheter. The axial strands can also be placed into compression to forcibly reduce the diameter of the introducer sheath and bring it into close approximation with the interventional catheter. Maintaining the introducer sheath in the smallest diameter configuration that will accommodate passage of the interventional catheter will result in reduced trauma to the access site due to reduced arterial access site stretch. This will allow the access site into the blood vessel to be sealed more readily following completion of the interventional procedure and removal of the sheath. Removal of the sheath can be accomplished by reducing its diameter over time thereby allowing the blood vessel tissues at the access site an opportunity to return to a smaller opening diameter.

In an alternate embodiment the introducer can be designed such that its equilibrium or relaxed state is in the larger diameter and activation of the strands under compression can occur to lengthen the introducer sheath and cause the diameter of the introducer to reduce for insertion into the vessel. Release of the strands after the introducer sheath is in position will allow the diameter to enlarge to the larger equilibrium configuration. The axial fibers can further be placed into tension to forcibly place the introducer into its large diameter configuration and hold it in this configuration. A large profile interventional catheter can then be passed through the introducer.

The expandable introducer can be introduced into the vessel with a smaller diameter conformation than a standard fixed diameter introducer sheath. This allows the vessel wall to undergo a direct radial expansion of the hole at the access site made by the needle puncture. The sliding friction provided by the larger dilator and fixed diameter introducer sheath of the standard method has been replaced by an improved radial expansion of the vascular access site. A smaller diameter dilator could therefore be used with the expandable introducer of the present invention. The transition between the dilator and the introducer sheath could include a transition ledge to ensure a smooth entry of the introducer into the blood vessel. Alternately, a small recess or pocket can be formed at the end of the dilator that would allow the distal end of the introducer sheath to be held in a smaller diameter configuration.

In still another embodiment an introducer is constructed such that its distal region which extends within the blood vessel is formed of a porous material such as the braided fibers with openings between the braid wires and no polymer coating. The proximal portion of the introducer that extends through the vessel wall at the access site has the braided wall structure coated with a polymer or extruded with polymer to provide for a seal between the introducer catheter and the vessel wall. Blood flow within the vessel is then provided a passage through the open braided structure of the distal introducer portion. The distal end of the tubular structure formed of braided fibers would be coated with a flexible polymeric coating to minimize vessel trauma due to the ends of the braided fibers. This flexible coating could extend from 0.5 mm to 5 mm beyond the distal end of the tubular structure.

The present invention also includes methods for forming the expandable introducer sheath. In one embodiment the axial fibers follow an interleaved path wherein they wind in an out in a radial direction between braided fibers as they extend generally axially from one end to the other of the braided introducer sheath. The interleaving of the axial strands within the braid provides the axial strands with support along a portion of their surface to allow them to transmit compression forces from the proximal end to the distal end of the introducer sheath. This interleaving of the axial strands within the braided fibers is an inward and outward weaving of the axial strands through the wall of the braid as the axial strands extend in a generally axial direction.

In one embodiment the axial strands are contained within a flexible polymeric coating that covers the braided fibers and the axial strands. This polymeric coating can be applied via an extrusion operation, a dip or spray coating operation, via application of a polymeric sheet or tubular wrap to the tubular braid structure and the axial strands, or via another method. To ensure that the axial strands are able to move axially under tension or compression they can be coated with a release agent such as a soap or other dissolvable material or placed with clearance within a flexible small tubular body that is itself covered by the flexible polymeric coating. The axial strands can also be directed generally axially along the inner or outer surface of the tubular structure or through the polymeric coating along with a containment means for providing support for the axial strands.

The axial strands can be either round or formed from a rectangular cross-section. The rectangular cross-section allows the axial strands to provide reduced profile to the tubular structure by placing the small dimension of the rectangular cross-section in the radial direction. The material for the axial strands can be metal such as stainless steel, nitinol, or other metal or alloy. A high modulus polymeric material could also be used for the axial strands, such as polyethylene terephthalate (PET), nylon, or other high modulus polymer used in medical catheters and devices. The strands would provide greater compression force in a monofilament strand than a multifilament strand.

The braided fibers can be formed from either monofilament or multifilament fibers that are braided at angles that optimize effectiveness of specific features. For example, braiding at a large angle with respect to the axis will provide the introducer sheath with a large outward force in its expanded configuration as the axial strands are placed under tension.

One embodiment of the invention has a large diameter equilibrium configuration and a large braid angle with respect to the axis. This introducer sheath can be placed into a vessel at the larger diameter configuration and remain approximately at this larger diameter. Axial strands placed into tension can cause this introducer sheath to become more rigid and help to straighten out tortuous vessels without significant changes to the diameter of the introducer sheath.

Braiding at a small angle with respect to the axis will provide the introducer sheath with a large axial extensional force with the axial strands placed under compression. The braided fibers can be formed of metal or polymeric materials typically used in interventional catheters or devices.

### BRIEF DESCRIPTION OF THE DRAWINGS

FIG. 1A is a plan view of a passively expandable introducer sheath in a smaller diameter configuration.

FIG. 1B is a plan view of a passively expandable introducer sheath in a larger diameter configuration.

FIG. 2A is a partially sectioned view of an actively expandable introducer sheath showing the inner braided structure and axial strands in a smaller diameter configuration.

FIG. 2B is a partially sectioned view of an actively expandable introducer sheath showing the inner braided structure and axial strands in a larger diameter configuration.

FIG. 2C is an isometric view of a portion of a rectangular axial strand.

FIG. 3A is a partially sectioned view of the expandable introducer sheath introduced into a tortuous vessel in its smaller diameter configuration.

FIG. 3B is a partially sectioned view of the expandable introducer sheath introduced into a tortuous vessel and expanded to its larger diameter configuration and having an interventional catheter contained within

FIG. 4A is a partially sectioned view of a dilator with a transition ledge and having an expandable introducer loaded upon it.

FIG. 4B is a partially sectioned view of a dilator with a recess and having an expandable introducer loaded upon it.

### DETAILED DESCRIPTION OF THE INVENTION

The present invention is an introducer sheath intended for providing access for passage of interventional catheters into blood vessels or other tubular vessels of the body. In vessels that are tortuous such as iliac arteries or other vessels of the leg, the introducer catheter will assist in straightening out the vessel. Also, this invention is intended to provide a smaller access site opening to the blood vessel as the introducer sheath is delivered into the blood vessel. The present introducer sheath is delivered at a smaller size than a standard introducer catheter. Further, the invention allows the introducer sheath to be reduced in diameter after it has been placed into the vessel by coming into close approximation with the wall of the interventional catheter. Also, the introducer sheath allows the access site to slowly reduce in diameter to a smaller diameter prior to removal of the introducer sheath and thereby assist in improving the vascular sealing of the access site.

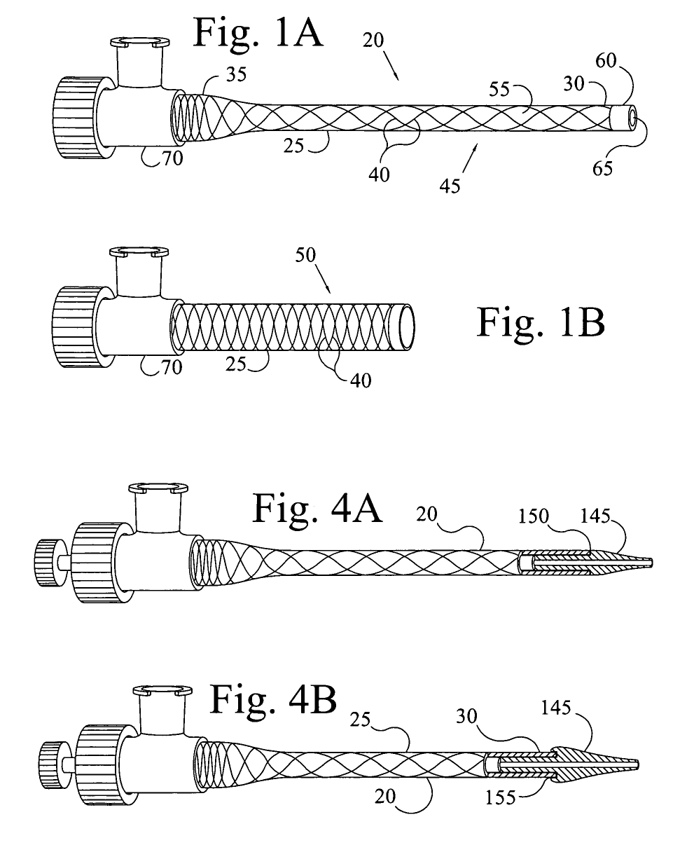

In one embodiment shown in FIGS. 1A and 1B the introducer sheath (20) is a tubular structure (25) having a distal end (30) and a proximal end (35) and formed with braided fibers (40). The introducer sheath (20) is placed into a blood vessel or other body vessel in its smaller equilibrium or relaxed state having a smaller diameter configuration (45) as shown in FIG. 1A. As a larger aspect of an interventional catheter is advanced through the introducer sheath (20), it passively expands out to a larger diameter configuration (50) as shown in FIG. 1B to provide passage for the larger aspect. The interventional catheter can be a therapeutic catheter, a diagnostic catheter, or a guide catheter. The distal aspect can contain a therapeutic device that requires a somewhat larger profile.

The braided fibers (40) can be formed of a metallic monofilament or multifilament strand such a stainless steel, nitinol, or other metal used in the medical device industry. The braided fibers (40) can also be formed from polymer materials such as polyethylene terephthalate, nylon, or other high strength polymeric materials used in the medical device industry. A polymeric coating (55) is located between the braided fibers (40) along the length of the tubular structure (25) providing a continuous polymeric layer. A polymeric material should preferably extend a small distance distally to the braided fibers (40) forming a polymeric soft tip (60) to the catheter. The polymeric coating (55) and the polymeric soft tip (60) should be formed of a material that can flex and stretch easily so that it can allow the diameter to enlarge as the length of the introducer is reduced due to the braided structure. Such polymeric coating (55) and polymeric soft tip (60) materials include polyurethane, Pebax, polyvinylchloride, silicone, or other polymers or copolymers used for flexible medical devices. A lubricious coating can be applied to the inner surface (65) of the introducer sheath (20) to reduce the friction with respect to the interventional catheter that moves within it. The lubricious coating can be a silicone liquid coating or it can be a thin film such as a fluoropolymer, hydrogel, or other lubricious film used in the medical device industry. Without this lubricious coating the introducer sheath (20) of this embodiment could tend to clamp down on the shaft of an interventional catheter that passes through it. A manifold (70) attached to the proximal end (35) of the tubular structure (25) provides passage and sealing with respect to interventional catheters and also allows for injection of fluids into the introducer.

The expandable introducer sheath (20) can also be configured with a larger equilibrium or relaxed state as shown in FIG. 1B and can be introduced into the body in a smaller diameter configuration as shown in FIG. 1A. The introducer sheath (20) can be placed over a dilator (145) as shown in FIG. 4B for introduction into the vasculature. The distal end (30) of the introducer sheath (20) can be held by recess (155) such that the expandable introducer is held in tension in a smaller diameter configuration. The smaller diameter configuration will allow the expandable introducer to pass more easily through tortuous or stenotic vasculature and provide passage for larger interventional catheters there through. Release of the introducer sheath (20) from the recess (155) will allow the introducer sheath to expand out to a larger diameter configuration.

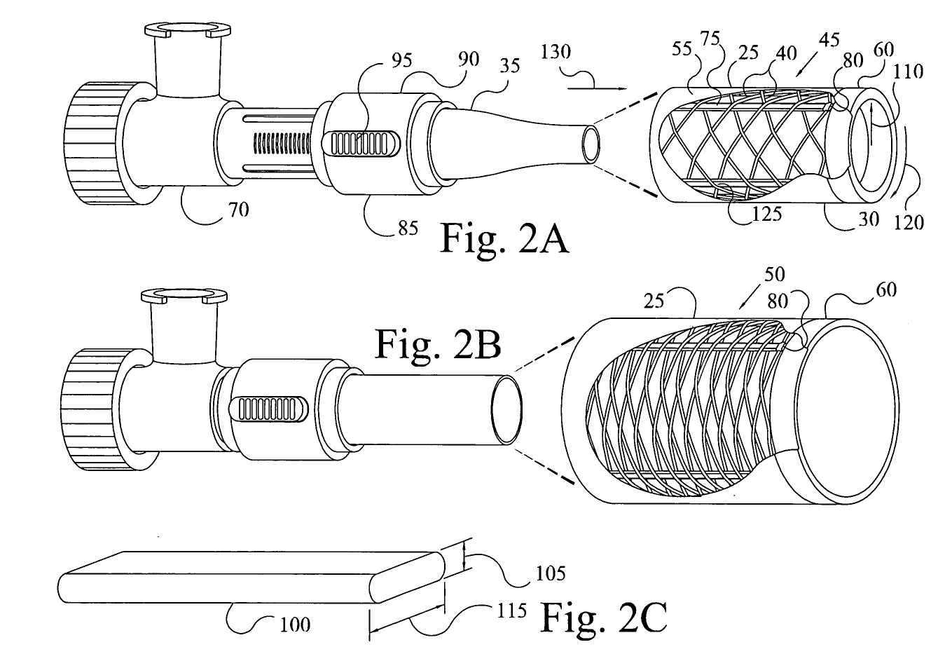

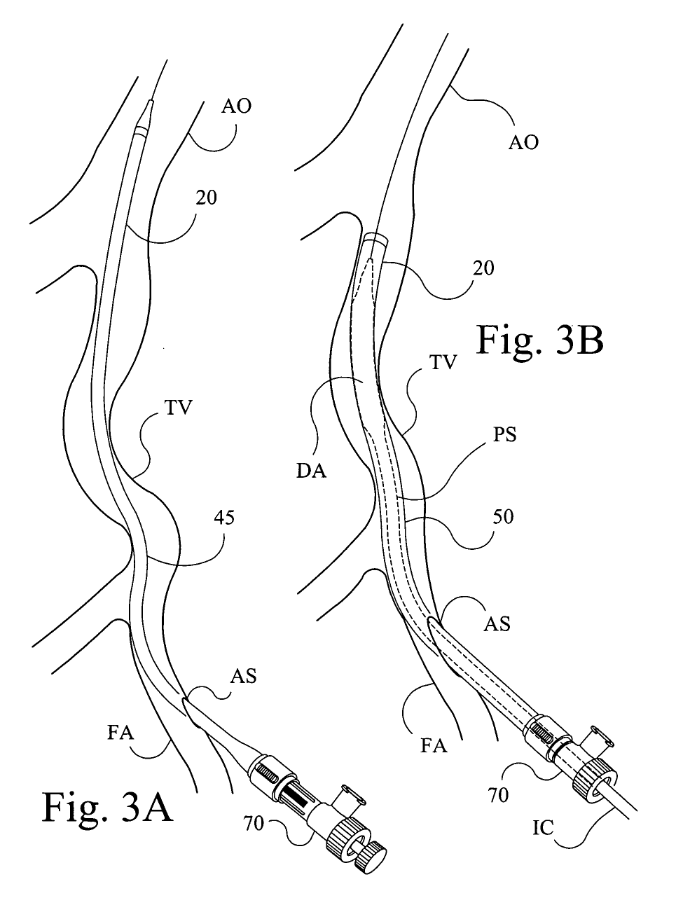

In a preferred embodiment of the invention the introducer sheath (20) shown in FIGS. 2A and 2B can be actively expandable by placing axial strands (75) within the wall or adjacent to the wall and attaching them to the distal end (30) of the braided fibers (40) at the strand attachments (80). In one embodiment the axial strands (75) are placed into compression to ensure the tubular sheath is expanded in length and reduced in diameter during insertion into an access site (AS) to a blood vessel such as the femoral artery (FA) as shown in FIG. 3A. After insertion into the blood vessel at the access site, application of tension to the strands in a proximal direction is provided to cause the length of the introducer to decrease and its diameter will increase by a factor of 20 percent to 300 percent. For example, a 9 French expandable introducer sheath (20) could expand to accommodate an 18 French interventional catheter.

The number of axial strands (75) could range from two to more than ten. If the expandable introducer sheath (20) is in its relaxed condition in its smaller diameter configuration (45) as shown in FIG. 2A, then compression of the axial strands (75) by activating the compression element (85) stabilizes the shape in the elongated and smaller diameter configuration (45). Application of tension to the strands via a tensioning element (90) will move the axial strands (75) and will cause the diameter to increase to a larger diameter configuration (50) as shown in FIG. 2B. A manifold (70) having a holding member (95) would hold the strands stationary in either tension or compression and hence hold the expandable introducer sheath (20) in its larger diameter (50) or smaller diameter (45) configuration, respectively.

The axial strands could be formed from a high extensional strength polymer such as polyethylene terephthalate (PET), Dacron, nylon, pebax, or other polymeric material. The axial strands (75) are preferably formed from a thin strand of a metal such as stainless steel, nitinol, or other metal or alloy. The axial strands (75) could be formed from a metal strand that has a cross-section that is round, square, or rectangular in shape. A rectangular axial strand (100) shown in FIG. 2C provides an advantage such that placing the smaller dimensional thickness (105) of the rectangle in the direction of the wall thickness, i.e., the radial direction (110), affords the introducer sheath (20) with a minimal profile while maximizing the strength of the axial strand. The width (115) of the rectangular axial strand (100) is directed in the circumferential direction (120) as shown in FIG. 2A.

The axial strand can be interleaved (125) in an axial direction (130) within the braided fibers (40) of the tubular structure (25) extending from the proximal end (35) to the distal end (30) as shown in FIGS. 2A and 2B. This interleaving of the axial strands (75) provides support to the axial strands (75) from the braided fibers (40) such that when they are placed into compression, they will transmit the force to the distal end (30) of the tubular structure (25) where the axial strands (75) are attached to the braided fibers (40) at the strand attachment. A metal rectangular axial strand (100) could have a width (115) ranging from 0.012 to 0.004 inches and a thickness (105) that ranges from 0.001 to 0.006 inches. Preferably the dimension of the width (115) and thickness (105) of a metal rectangular axial strand (100) is approximately 0.007 by 0.003 inches, respectively.

A polymeric coating (55) is located between the braided fibers (40) as has been described in FIG. 1A. In a preferred embodiment the polymeric coating (55) is a continuous coating that extends along the entire length of the tubular structure (25). At the distal end (30) of the tubular structure (25) a soft tip (60) that is formed of flexible polymeric material extends beyond the ends of the braided fibers (40) to ensure an atraumatic distal end (30) to the introducer sheath (20).

In an alternate configuration, the polymeric coating can extend only along a proximal portion of the introducer sheath and the braided fibers remain uncoated in the distal portion. The open braided structure at the distal portion allows blood to flow through the open structure. The axial strands can also extend entirely on the outside or inside of the braided fibers and can be contained within the polymeric coating for support. Various techniques can be applied to ensure ease of movement for the axial strands within the polymeric coating. For example, the axial strands can be contained within a flexible tubing that affords the strands clearance and support. The flexible tubing can be contained within the polymeric coating provided that movement relative to the polymer coating is maintained. The axial strands can be coated with a slip or release agent to allow freedom of movement within the polymeric coating. Such slip or release agents include silicone oils, hydrogels, polytetrafluoroethylene, and other agents commonly applied to interventional catheters to provide for relative movements.

To use this introducer sheath, one could introduce it along with a dilator over a guidewire in its smaller diameter and flexible state. The diameter of the introducer sheath during insertion is intended to be smaller than that for a standard fixed diameter introducer sheath. Placing the axial strands (75) under compression via the compression element (85) and holding the compression via the holding member (95) located on the manifold (70) would ensure that the introducer sheath (20) remains in a longer length and smaller diameter configuration (45) as shown in FIGS. 2A and 3A. Once inside the blood vessel or other tubular vessel the axial strands could be placed into tension via the tensioning element (90) and held via the holding member (95) to hold the introducer in its larger diameter configuration (50) as shown in FIG. 3B. The larger diameter configuration (50) shown in FIG. 2B places the braided fibers (40) into close approximation with each other and places the introducer sheath (20) into a more rigid large diameter configuration that will help to straighten out tortuous vessels (TV) as shown in FIG. 3B. The introducer sheath (20) can be extended, for example, from the femoral artery (FA) into the aorta (AO) to provide passage for the interventional catheter (IC) as shown in FIG. 3B. If a lesion is present within the blood vessel or tubular vessel of the body that causes a narrowing of the introducer sheath (20), a balloon dilatation procedure can be performed within the introducer sheath (20) to enlarge the narrowing.

Introduction of the present expandable introducer sheath (20) into the blood vessel in a smaller configuration as shown in FIG. 3A will generate less trauma to the blood vessel at the access site. This smaller diameter configuration (45) is smaller than the diameter of a standard introducer sheath (20). The expansion of the introducer as shown in FIGS. 2B and 3B is a radial expansion with less trauma to the access site (AS) of the blood vessel than the excessive sliding friction caused by a standard fixed diameter introducer and dilator. A standard dilator and introducer will create a focusing of expansion on local weaker regions of the vessel wall which can then tear. Vascular closure with the expandable introducer sheath (20) following the procedure will likely be improved due to the more uniform and gentle radial expansion of the access site.

An interventional catheter (IC) with a larger distal aspect (DA) can be easily passed through the introducer as shown in FIG. 3B. Once the distal end (30) of the interventional catheter has passed through the introducer sheath (20), the axial strands that were held under tension can be released and the introducer reduces in diameter similarly to that shown in FIG. 3A and comes to rest at a smaller diameter that allows free passage of the proximal shaft (PS) of the interventional catheter (IC). The access site of the blood vessel is reduced in size by lowering the diameter of the introducer such that minimal clearance is provided to the interventional catheter. If necessary, compression can be applied to the axial strands (75) to help place the introducer sheath (20) into close approximation with the smaller shaft body of the interventional catheter.

The holding member (95) located on the manifold (70) shown in FIG. 2A can hold the axial strands (75) in various positions that control the diameter of the expandable introducer sheath (20). Prior to removal of the expandable introducer sheath (20), the diameter of the introducer sheath (20) can be reduced incrementally to a smaller diameter to allow the vessel opening to relax back to a smaller dimension. Removal of the introducer sheath (20) after allowing the access site (AS) of the vessel to relax will reduce the amount of time necessary to accomplish vascular closure at the access site.

The dilator (145) used with this embodiment or other embodiments of this invention can have a transition ledge (150) as shown in FIG. 4A. The transition ledge (150) can form a recess (155) as shown in FIG. 4B to provide a pocket to contain the distal end (30) of the tubular structure (25) during insertion into the body. The recess (155) serves to ensure that the distal end (30) of the tubular structure (25) cannot enlarge in diameter until the introducer sheath (20) has been positioned within the blood vessel. The transition ledge (150) can form a smooth transition from the dilator (145) to the introducer to provide for a smooth insertion into the blood vessel. Once inside the blood vessel, expansion of the introducer will provide clearance for the transition ledge (150) and allow the dilator (145) to be withdrawn proximally out of the blood vessel.

As an alternate embodiment the introducer sheath (20) can be structured such that its equilibrium state is its expanded state with a larger diameter configuration (50) as shown in FIG. 2B and the axial strands (75) are then placed under compression via the compression element (85) and held via the holding member (95) to hold the introducer in its smaller diameter configuration (45) as shown in FIG. 2A for placement within the vessel. Release of the axial strands (75) would then allow the introducer to assume its larger diameter configuration (50). Activation of the tensioning element (90) and holding member (95) would further ensure that the introducer sheath (20) is expanded into its larger diameter configuration (50) after it is positioned within the blood vessel.

In another embodiment the introducer sheath (20) can be formed with an equilibrium larger diameter configuration (50) similar to that shown in FIG. 2B and with braided fibers (40) having a braid angle with respect to the axis of almost 90 degrees. This introducer sheath (20) can be introduced into a blood vessel in this large diameter configuration with a relatively flexible tubular structure (25) with the axial strands (75) not under significant tension or compression by not activating the tensioning element (90) or compression element (85). It would be able to extend easily through a toutuous vessel that was large enough to accept this diameter of introducer sheath (20). Once the introducer sheath (20) is in position through the tortuous vessel, the axial strands (75) can be placed into tension by activating the tensioning element (90) to cause the introducer sheath (20) to become more rigid as the tubular structure (25) is held in its fully enlarged diameter configuration and help straighten out the blood vessel. An interventional catheter can then be allowed passage through the introducer sheath (20). This introducer sheath (20) has undergone minimal or no diameter change during its use.

### REFERENCE NUMERALS

20 Introducer Sheath
25 Tubular Structure
30 Distal End
35 Proximal End
40 Braided Fibers
45 Smaller Diameter Configuration
50 Larger Diameter Configuration
55 Polymeric Coating
60 Soft Tip
65 Inner Surface
70 Manifold
75 Axial Strands
80 Strand Attachments
85 Compression Element
90 Tensioning Element
95 Holding Member
100 Rectangular Axial Strand
105 Thickness
110 Radial Direction
115 Width
120 Circumferential Direction
125 Interleaved Fibers and Strands
130 Axial Direction
145 Dilator
150 Transition Ledge
155 Recess

1. An introducer sheath that is delivered percutaneously into the access site of a blood vessel and positioned within a blood vessel for providing passage for another interventional catheter there through, said introducer sheath comprising;
A. A tubular structure having braided fibers extending there through, said tubular structure able to be expanded in diameter from its proximal end to its distal end,
B. More than one axial strands extending through the wall of said tubular structure from the proximal end and attaching to the distal end of said tubular structure,
C. Said introducer sheath having a tensioning means to provide tension to said axial strands,
D. Said introducer having a holding means at the proximal end to hold the proximal ends of said axial strands to prevent their axial movement,
E. Said tubular structure having a continuous flexible polymeric coating applied along at least a portion thereof, wherein said introducer sheath is introduced into the blood vessel in a smaller diameter relaxed configuration and said axial strands are placed under tension via said tensioning means to expand the entire length of said tubular structure out to a larger diameter configuration after positioning within the blood vessel.

2. The introducer sheath of claim 1 wherein said introducer sheath further comprises a compression means to place said axial strands into compression during delivery, said holding means preventing axial movement of said axial strands during delivery to the blood vessel, thereby preventing diametric enlargement of the introducer sheath during delivery.

3. The introducer sheath of claim 1 wherein said braided fibers are in close approximation to each other upon expansion of said tubular member to a larger diameter configuration thereby providing said introducer sheath with greater rigidity and ability to straighten out a tortuous blood vessel.

4. The introducer sheath of claim 2 wherein said axial strands are interleaved between said braided fibers from said proximal end to said distal end of said tubular member thereby constraining said fibers along a portion of their surface.

5. The introducer sheath of claim 1 wherein said axial strands are fonned of a rectangular cross-section for maximal compressive and tensile strength to provide for a minimal introducer sheath wall profile.

6. The introducer sheath of claim 2 wherein said axial fibers are placed into compression with said compression means following placement of the introducer sheath into the blood vessel thereby causing said tubular structure to reduce in diameter to provide minimal clearance to the interventional catheter and thereby provide minimal stretching to the access site by the introducer sheath.

7. The method of use for an introducer sheath having a braided tubular structure that is delivered percutaneously into an access site of a blood vessel for providing passage for another interventional catheter there through, said introducer sheath having axial strands extending from the proximal end and attaching to the distal end of said tubular structure, and having a tensioning means to provide tension to said axial strands, a compression means to place said axial strands into compression, and a holding means to hold said axial strands and prevent their axial movement, said method comprising the steps;
A. introducing said introducer sheath into the blood vessel in a smaller relaxed diameter configuration,
B. placing said axial strands into tension to cause the tubular structure to enlarge in diameter thereby straightening out a tortuous blood vessel and stretching the access site into the blood vessel with a radial stretching motion,
C. entering the introducer with an interventional catheter and passing it through the introducer sheath for intervention.

8. The method of claim 7 further comprising the steps of activating the compression means and holding means to reduce the diameter of the introducer sheath, thereby placing the introducer sheath into close approximation with the interventional catheter and providing reduced stretch to the access site of the blood vessel.

9. The method of claim 8 further comprising the steps of removing the interventional catheter and reducing the diameter of the introducer sheath gradually over time to allow the access site to reduce in diameter slowly over time.

10. The introducer sheath of claim 1 wherein said introducer sheath further comprises a dilator sheath over which it is placed during delivery into the blood vessel; said dilator sheath having a transition ledge.

11. The introducer sheath of claim 10 wherein said dilator sheath further has a recess pocket that is positioned over the distal end of said tubular structure of said introducer sheath during delivery into the blood vessel.
